(19) United States
(12) Patent Application Publication
(10) Pub. No.: US 2005/0273025 A1
(43) Pub. Date: Dec. 8, 2005

(54) Braces having an assembly for exerting a manually adjustable force on a limb of a user

(76) Inventor: Guy M. Houser, Bainbridge Island, WA (US)

Correspondence Address: PERKINS COIE LLP;PATENT-SEA, P.O. BOX 1247, SEATTLE, WA, 98111-1247, US

(21) Appl. No.: 11/130,889

(22) Filed: May 17, 2005

Related U.S. Application Data
(60) Provisional application No. 60/572,894, filed on May 19, 2004.

(51) Int. Cl. A61F 5/00

(52) U.S. Cl. 602/16; 602/5

(57) ABSTRACT

Braces having an assembly for exerting a manually adjustable force on a limb of a user are disclosed herein. In one embodiment, a brace includes a first frame portion, a second frame portion, a hinge movably coupling the first frame portion to the second frame portion, a flexible member positioned relative to the first and/or second frame portion for exerting a force on the limb of the user, and a tensioning mechanism for manually adjusting a tension in the flexible member to vary the force exerted on the limb of the user. The force exerted by the flexible member is generally independent of the position of the first frame portion relative to the second frame portion.

### CROSS-REFERENCE TO RELATED APPLICATION

This application claims the benefit of U.S. Provisional Application No. 60/572,894, filed on May 19, 2004, entitled “Braces Having an Assembly for Selectively Exerting a Force on a Limb of a User,” which is incorporated by reference herein.

### TECHNICAL FIELD

The present invention is related to braces having an assembly for exerting a manually adjustable force on a limb of a user.

### BACKGROUND

Knee braces are widely used to stabilize and protect the knee joint. For example, knee braces are often used to prevent damage to the anterior cruciate ligament, posterior cruciate ligament, medial collateral ligament, lateral collateral ligament and/or meniscus in a knee joint. Knee braces are particularly useful to protect the knee joint during vigorous athletic activities such as running, basketball, football and skiing, and they are also used to stabilize the knee joint during recovery or rehabilitation from surgery or an injury.

A knee brace typically includes an upper frame, a lower frame, and a hinge connecting the upper frame to the lower frame. The upper frame often has straps that wrap around the quadriceps or hamstring, and the lower frame often has straps that wrap around the calf. Each portion of the frame is configured to fit the shape of the corresponding portion of the leg. The hinge allows the lower frame to pivot relative to the upper frame as the knee bends. Many braces have a hinge on each side of the knee joint to give the brace additional strength.

Some conventional knee braces are designed to provide additional support to different portions of the knee joint. For example, several knee braces provide support to the tibial condyles by applying a force with a static strap or a rigid frame. Braces having static straps for applying a force, however, have several disadvantages. First, users must typically remove the brace to adjust the strap and change the force because it is difficult to tighten the strap while the brace is on the leg. Second, it is sometimes an iterative process to adjust the strap to the precise location for exerting a desired force. Specifically, users often end up removing the brace, adjusting the strap to change the force, donning the brace back onto the leg, determining if the adjusted force is the desired force, and repeating the process until the strap is positioned to apply the desired force. Thus, adjusting the strap to provide a desired force in conventional knee braces can be a hassle and time consuming process.

Other knee braces include devices for providing dynamic forces to support the knee joint. For example, one conventional knee brace includes an upper frame, a lower frame moveably coupled to the upper frame, two pulleys attached to the lower frame, and a steel wire. The steel wire has a first end attached to one side of the upper frame, a second end attached to the other side of the upper frame, and an intermediate portion extending around the two pulleys such that a section of the wire between the two pulleys is positioned over the tibia. The movement of the upper frame relative to the lower frame changes the tension in the wire. Specifically, when the brace is at the full-extension position the wire is taut and exerts a force on the tibia, and when the brace is at the full-flexion position the wire has slack and does not exert a force on the tibia.

### BRIEF DESCRIPTION OF THE DRAWINGS

FIG. 1 is a rear view of a portion of a knee brace in accordance with one embodiment of the invention.

FIG. 2 is an exploded front isometric view of the tensioning mechanism.

FIG. 3 is a front isometric view of a portion of the tensioning mechanism.

FIG. 4 is a front isometric view of a drive shaft, a center gear, peripheral gears, and a spool of the tensioning mechanism assembled together.

FIG. 5 is a side cross-sectional view of the drive shaft, the center gear, the peripheral gears, and the housing assembled together.

FIG. 6 is a front isometric view of a portion of the tensioning mechanism including a housing, a clutch received in the housing, and a clutch hex received in the clutch.

FIG. 7 is a front isometric view of the clutch hex received in a recess of the housing.

### DETAILED DESCRIPTION

A. Overview

The present invention is related to braces having an assembly for exerting a manually adjustable force on a limb of a user. In one embodiment, a brace includes a first frame portion, a second frame portion, a hinge movably coupling the first frame portion to the second frame portion, a flexible member positioned relative to the first and/or second frame portion for exerting a force on the limb of the user, and a tensioning mechanism for manually adjusting a tension in the flexible member to vary the force exerted on the limb of the user. The force exerted by the flexible member is generally independent of the position of the first frame portion relative to the second frame portion.

In one aspect of this embodiment, the flexible member is positioned to exert the force over a “T” shaped area of the limb of the user. The flexible member can be a cable, filament, and/or other suitable member that can withstand the operating tensile loads of the brace. The tensioning member can include a roller for winding the cable and/or filament, a driving member for rotating the roller in a first direction, and a clutch for selectively inhibiting rotation of the roller in a second direction opposite the first direction.

In another embodiment, a knee brace for exerting a force on a tibia of a user includes an upper frame, a lower frame, a hinge movably coupling the lower frame to the upper frame, first and second guides attached to the lower frame, a cable and/or filament having a segment extending between the first and second guides, and a tensioning mechanism attached to the lower frame. The segment of the cable and/or filament is positioned to selectively exert a force on the tibia of the user. The tensioning mechanism includes a driving member for manually adjusting the tension in the cable and/or filament to change the force exerted on the tibia of the user. The first and second guides can include first and second pulleys around which the cable and/or filament pass.

In another embodiment, a brace for use on a limb of a user includes a first frame portion, a second frame portion, a hinge movably coupling the first frame portion to the second frame portion, a cable and/or filament positioned relative to the first and/or second frame portion for exerting a force on the limb of the user, and means for manually adjusting a tension in the cable and/or filament. The means for manually adjusting the tension can include a manually adjustable driving member coupled to the cable and/or filament for adjusting the tension in the cable and/or filament. Moreover, the means for manually adjusting the tension are configured so that a user can change the force without manipulating a strap on the brace or removing the brace from the limb.

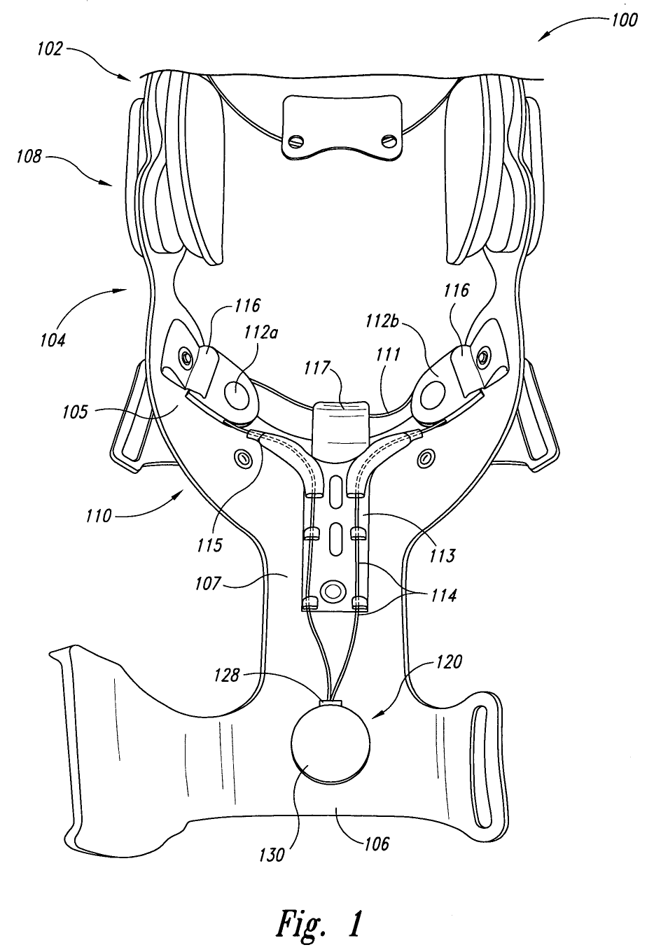

The following disclosure describes several embodiments of knee braces having assemblies for exerting a force on a limb of a user and methods for operating such braces. Several details describing structures or processes that are well known and often associated with other types of braces are not set forth in the following description for purposes of brevity. Moreover, although the following disclosure sets forth several embodiments of different aspects of the invention, several other embodiments of the invention can have different configurations or different components than those described in this section. As such, it should be understood that the invention may have other embodiments with additional elements or without several of the elements described below with reference to FIGS. 1-7. For example, even though many embodiments of assemblies for exerting a force are described below with reference to knee braces, they can also be used in elbow braces and other braces.

B. Embodiment of Knee Braces

FIG. 1 is a rear view of a portion of a knee brace 100 in accordance with one embodiment of the invention. The knee brace 100 includes an upper frame 102 (only a small portion of which is shown), a lower frame 104, and a hinge 108 coupling the upper frame 102 to the lower frame 104. The upper and lower frames 102 and 104 can have various configurations and/or shapes. For example, the illustrated lower frame 104 includes a first lateral portion 105, a second lateral portion 106, and a medial portion 107 extending between the first and second lateral portions 105 and 106. The lower frame 104 can also include a plurality of straps (not shown in FIG. 1 to avoid obscuring aspects of the brace 100) to attach the brace 100 to a user. The hinge 108 can be a bicentric hinge, such as any one of the hinges disclosed in pending U.S. patent application Ser. Nos. 10/077,469 and 11/051,198, both of which are incorporated by reference herein. In other embodiments, the hinge 108 may not be bicentric, but may have another configuration.

The knee brace 100 further includes an assembly 110 for selectively exerting a manually adjustable force on a tibia of a user. The illustrated assembly 110 includes a cable 111, first and second guides or pulleys 112a-b for supporting the cable 111, a cable guide 113 for guiding the cable 111 relative to the brace 100, and a tensioning mechanism 120 for adjusting the tension in the cable 111. The cable 111 can be a steel cable, Kevlar strand, filament, monofilament, or other suitable member comprised of metallic, fibrous, synthetic, and/or other suitable materials to withstand operating tensile loads. The first and second pulleys 112a-b are attached to opposing sides of the first lateral portion 105 and oriented such that the segment of the cable 111 extending between the pulleys 112 is positioned proximate to the tibia of the user. Straps 116 can attach the first and second pulleys 112a-b to the first lateral portion 105 so that the pulleys 112 can move relative to the lower frame 104 and align themselves with the cable 111 as the cable 111 is tightened. The system 110 may also include a strap 117 for positioning the cable 111 at a proper location relative to the tibia when the cable 111 is tightened. In additional embodiments, the assembly 110 may not include two pulleys 112. For example, the assembly 110 may include straps in lieu of or in addition to pulleys for positioning the cable 111 proximate to the tibia.

The cable guide 113 is attached to the medial portion 107 with glue, rivets, or other suitable fasteners and guides the cable 111 between the tensioning mechanism 120 and the pulleys 112. The cable guide 113 can include a plurality of eyelets 114, conduits 115, and/or other members for guiding the cable 111 over the medial portion 107. Other embodiments may not have a cable guide 113, or may include a cable guide with a different configuration. The tensioning mechanism 120 allows a user to selectively adjust the tension in the cable 111 and change the force the cable 111 exerts on the tibia and is described in greater detail below. The knee brace 100 may also include one or more pads (not shown in FIG. 1 to avoid obscuring aspects of the brace 100) attached to the lower frame 104 and covering one or more of the components to increase the comfort of the brace 100. For example, the brace 100 may include a pad over the first and second pulleys 112a-b and the segment of the cable 111 between the pulleys 112 so that the cable 111 exerts a force on the tibia through the pad(s).

C. Embodiments of Tensioning Mechanisms

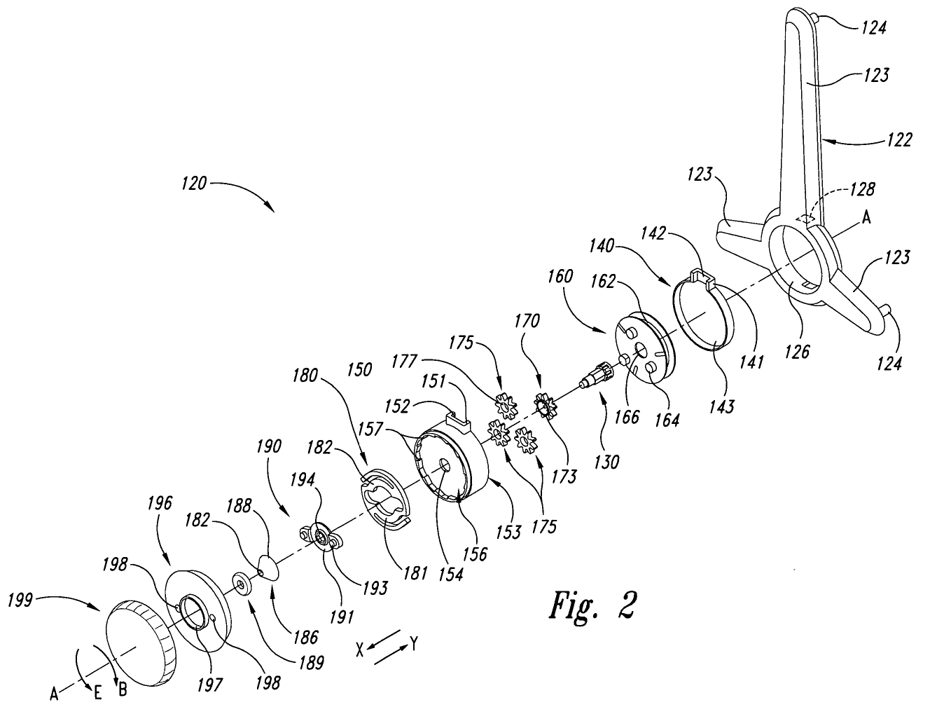

FIG. 2 is an exploded front isometric view of the tensioning mechanism 120 of FIG. 1. The illustrated tensioning mechanism 120 includes a mounting member 122, a bottom cap 140 received in the mounting member 122, a housing 150 attached to the bottom cap 140 and received in the mounting member 122, and a wheel or spool 160 enclosed between the bottom cap 140 and the housing 150. The illustrated mounting member 122 has a plurality of arms 123, a plurality of bosses 124 projecting from corresponding arms 123, and a recess 126 sized to receive the bottom cap 140 and the housing 150. The bosses 124 on the arms 123 can be inserted into corresponding apertures in the front surface of the medial portion 107 (FIG. 1) and/or the second lateral portion 106 (FIG. 1) to rigidly attach the mounting member 122 to the lower frame 104 (FIG. 1).

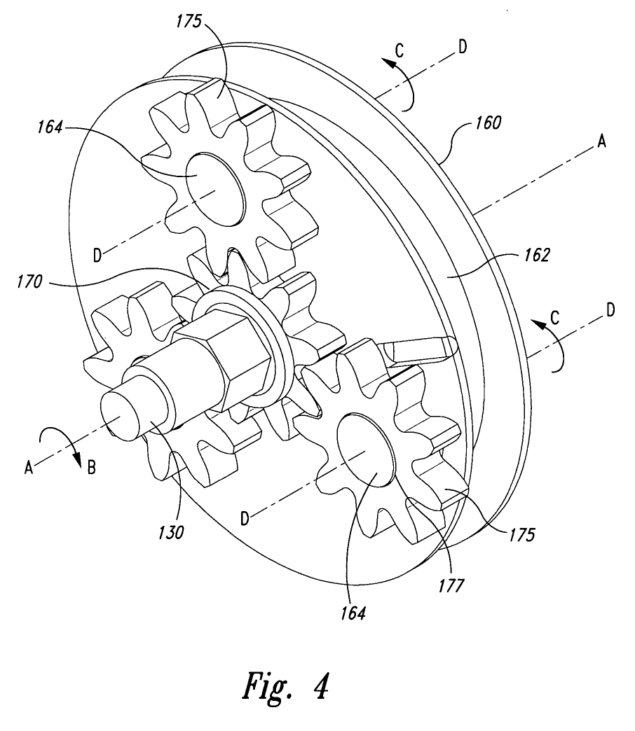

The bottom cap 140 includes a projection 141, a slot 142 in the projection 141, and a recess 143 sized to receive a portion of the spool 160. The housing 150 includes a projection 151, a slot 152 in the projection 151, a recess 153 (not shown in FIG. 2) sized to receive a portion of the spool 150, and a center aperture 154 defining an axis A-A. The spool 160 includes a groove 162 (best seen in FIG. 4) for receiving the cable 111 (FIG. 1), a center aperture 166 aligned with the aperture 154, and a plurality of bosses 164 projecting in a direction generally parallel to the axis A-A. When the bottom cap 140 is attached to the housing 150 and the spool 160 is received in the recesses 141 and 151, the projections 141 and 151 are aligned so that the cable 111 can extend from the spool 160 through the slots 143 and 153. The mounting member 122 also includes an aperture 128 (shown clearly in FIG. 1) sized to receive the projections 141 and 151 and through which the cable 111 passes.

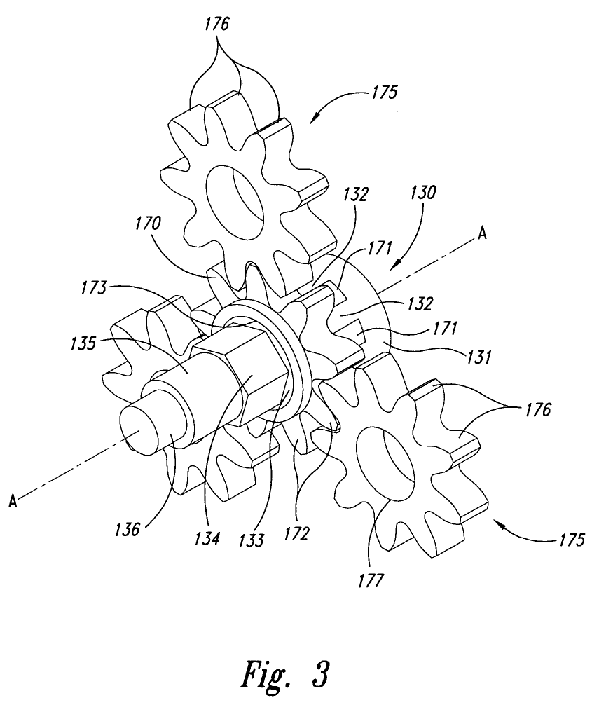

FIG. 3 is a front isometric view of a portion of the tensioning mechanism 120 including a drive shaft 130, a center gear 170, and a plurality of peripheral gears 175. The illustrated drive shaft 130 includes a first cylindrical portion 131 having a first diameter, a second cylindrical portion 133 having a second diameter less than the first diameter, a hexagonal portion 134, a third cylindrical portion 135 having a third diameter less than the second diameter, and a fourth cylindrical portion 136 having a fourth diameter less than the third diameter. The first diameter is sized so that the first cylindrical portion 131 can be received in the aperture 166 (FIG. 2) of the spool 160 (FIG. 2). The first cylindrical portion 131 also includes a plurality of teeth 132 projecting in a direction generally parallel to the axis A-A. The center gear 170 includes a first plurality of teeth 171 projecting in a direction generally parallel to the axis A-A, a second plurality of teeth 172 projecting radially outward, and a center aperture 173 sized to receive the second cylindrical portion 133 of the drive shaft 130. The first teeth 171 on the center gear 170 are configured to mesh with the teeth 132 on the drive shaft 130. The individual peripheral gears 175 include a plurality of teeth 176 configured to mesh with the second teeth 172 on the center gear 170 and an aperture 177 sized to receive a corresponding boss 164 (FIG. 2) of the spool 160.

FIG. 4 is a front isometric view of the drive shaft 130, the center gear 170, the peripheral gears 175, and the spool 160 assembled together. The first cylindrical portion 131 (FIG. 3) of the drive shaft 130 is received in the aperture 106 (FIG. 2) of the spool 160, and the bosses 164 are received in the apertures 177 of corresponding peripheral gears 175. When the teeth 132 (FIG. 3) on the drive shaft 130 and the first teeth 171 (FIG. 3) on the center gear 170 are engaged and the drive shaft 130 rotates about the axis A-A in a direction B, the drive shaft 130 rotates the center gear 170 about the axis A-A in the direction B, which in turn drives the peripheral gears 175 so that each peripheral gear 175 rotates in a direction C about a corresponding axis D-D defined by the respective boss 164.

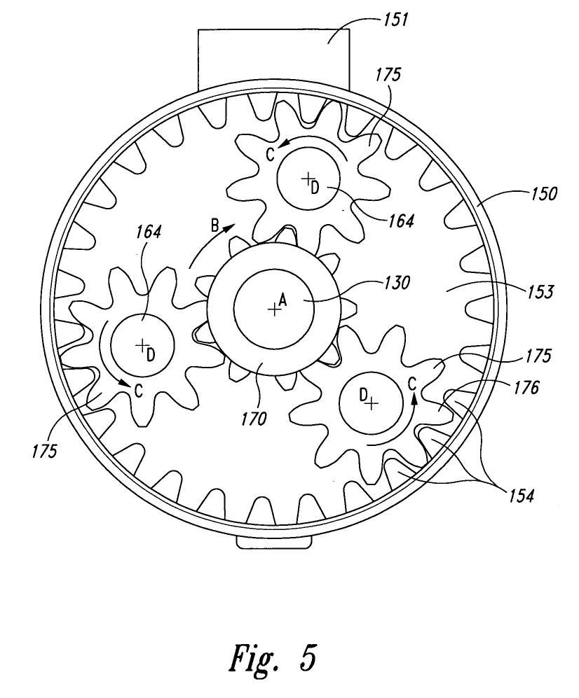

FIG. 5 is a side cross-sectional view of the drive shaft 130, the center gear 170, the peripheral gears 175, and the housing 150 assembled together. The housing 150 includes a plurality of teeth 154 projecting radially inward from an inner radial wall. The teeth 154 are configured to mesh with the teeth 176 on the peripheral gears 175. Referring to FIGS. 4 and 5, because the housing 150 is fixed and does not rotate relative to the mounting member 122 (FIG. 1), the rotation of the peripheral gears 175 in the direction C about the corresponding axes D-D drives the peripheral gears 175 and the bosses 164 around the axis A-A in the direction B. As such, when the teeth 132 (FIG. 3) of the drive shaft 130 and the teeth 171 (FIG. 3) of the center gear 170 are interlocked and the drive shaft 130 rotates in the direction B about the axis A-A, the drive shaft 130 drives the spool 160 about the axis A-A in the direction B.

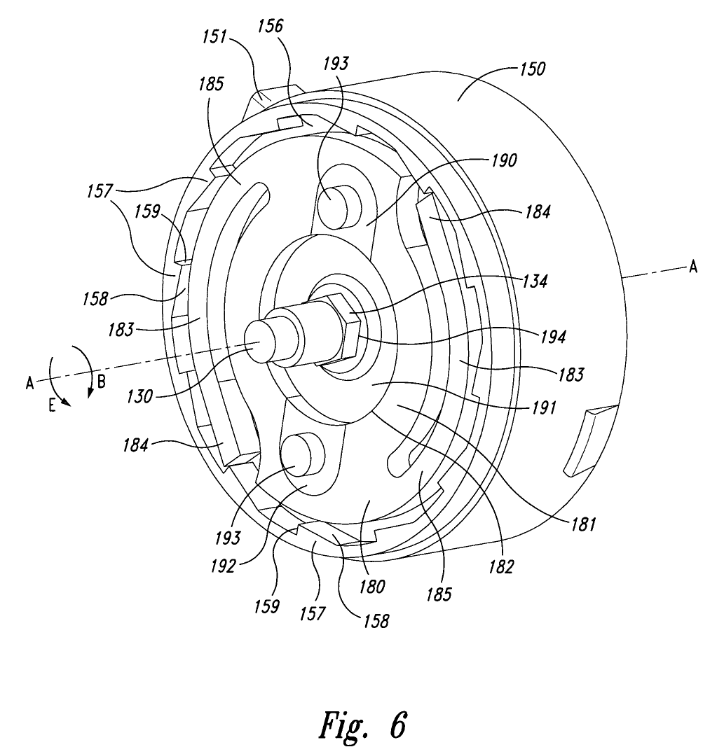

FIG. 6 is a front isometric view of a portion of the tensioning mechanism 120 including a clutch 180 received in the housing 150 and a clutch hex 190 received in the clutch 180. The housing 150 includes a recess 156 and a plurality of angled stops 157 projecting radially inwardly. The individual angled stops 157 have a ramp 158 and a step 159. The illustrated clutch 180 includes a body 181, an aperture 182 in the body 181 sized to receive the clutch hex 190, and a plurality of flexible members 183 projecting from the body 181. The flexible members 183 have a distal end 184, a proximal end 185, and a curvature between the distal and proximal ends 184 and 185 such that the distance between the distal end 184 and the drive shaft 130 is slightly greater than the distance between the proximal end 185 and the drive shaft 130. The flexible nature of the members 183 allows the clutch 180 to rotate in the direction B about the axis A-A because the members 183 flex radially inward as the distal ends 184 move across the ramps 158 of the stops 157. The flexible members 183, however, inhibit the clutch 180 from rotating about the axis A-A in a direction E opposite the direction B. Specifically, as the clutch 180 begins to pivot in the direction E, the distal end 184 of at least one of the flexible members 183 contacts the step 159 of a stop 157, which obstructs further rotation of the clutch 180. In additional embodiments, the clutch 180 can have other configurations, including a different number of flexible members 183.

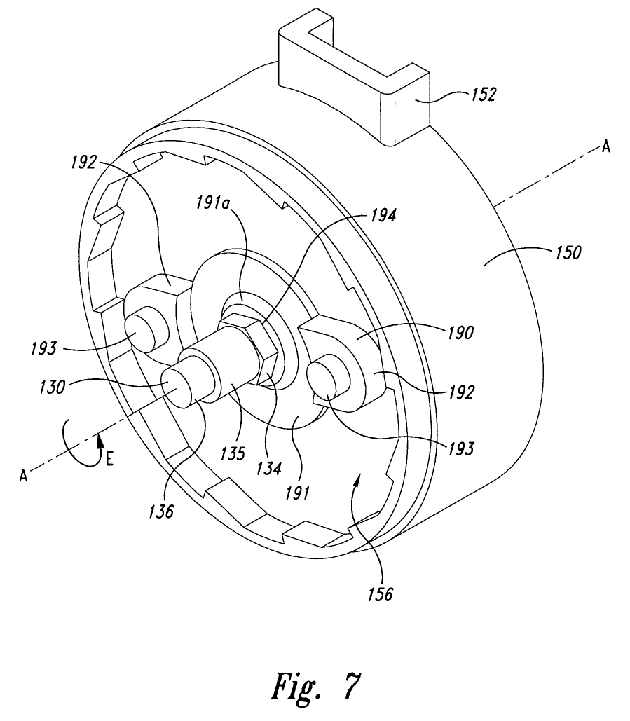

FIG. 7 is a front isometric view of the clutch hex 190 received in the recess 156 of the housing 150 (with the clutch 180 removed for clarity). The illustrated clutch hex 190 includes a base 191, a plurality of arms 192 projecting axially and radially outward, and a plurality of bosses 193 projecting from corresponding arms 192. Referring to both FIGS. 6 and 7, the arms 192 in the clutch hex 190 and the aperture 182 (FIG. 6) in the clutch 180 (FIG. 6) are configured so that when the clutch hex 190 is received in the clutch 180, the clutch hex 190 and the clutch 180 rotate together about the axis A-A. Accordingly, the flexible members 183 (FIG. 6) of the clutch 180 prevent the clutch hex 190 from rotating in the direction E about the axis A-A. The clutch hex 190 further includes a hexagonal aperture 194 sized to receive the hexagonal portion 134 of the drive shaft 130. As such, when the hexagonal portion 134 is positioned in the hexagonal aperture 194, the drive shaft 130 and the clutch hex 190 rotate together about the axis A-A.

Referring to both FIGS. 2 and 7, the tensioning mechanism 120 can further include a conical spring 186 (FIG. 2) for urging the drive shaft 130 in a direction X along the axis A-A and a retaining ring 189 for securing the conical spring 186 to the drive shaft 130. The conical spring 186 includes a first end 187 positioned over the third cylindrical portion 135 of the drive shaft 130 and a second end 188 positioned against the base 191 of the clutch hex 190. The clutch hex 190 may include a raised feature 191a on the base 191 for aligning the second end 188 of the conical spring 186. The raised feature 191a can have a diameter less than the diameter of the second end 188 so that the raised feature 191a is received in the second end 188. The retaining ring 189 can be attached to the fourth cylindrical portion 136 of the drive shaft 130 to retain the spring 186 on the drive shaft 130. Because the retaining ring 189 is attached to the drive shaft 130, the axial force exerted by the spring 186 urges the drive shaft 130 in the direction X along the axis A-A, which causes the teeth 132 (FIG. 3) of the drive shaft 130 to interlock with the first teeth 171 (FIG. 3) of the center gear 170 (FIG. 3).

Referring only to FIG. 2, the illustrated tensioning mechanism 120 further includes a knob 196 having a center aperture 197 and two radial apertures 198. The center aperture 197 is sized to receive the retaining ring 189 and the fourth cylindrical portion 136 (FIG. 6) of the drive shaft 130. The radial apertures 198 are sized and positioned to receive corresponding bosses 193 of the clutch hex 190. As such, rotation of the knob 196 is transmitted to the clutch hex 190. The tensioning mechanism 120 can also include a top cap 199 positioned over the knob 196.

D. Embodiments of Methods for Operating Tensioning Mechanisms

In operation, a user can change the tension in the cable 111 (FIG. 1) to adjust the force exerted against the tibia. For example, to increase the tension in the cable 111 and the corresponding force exerted against the tibia, the user rotates the knob 196 in the direction B. Rotation of the knob 196 drives the clutch hex 190 because the bosses 193 are positioned in corresponding apertures 198. The clutch hex 190 in turn rotates the drive shaft 130 due to the connection between the hexagonal aperture 194 and the hexagonal portion 134 (FIG. 6). The drive shaft 130 transmits rotation to the center gear 170 via the teeth 132 and 171 (FIG. 3), which drives the peripheral gears 175, which in turn rotate the spool 160. Rotation of the spool 160 winds the cable 111 (FIG. 1) and, consequently, increases the tension in the cable 111 to adjust the force exerted on the tibia.

A user can also reduce the tension in the cable 111 to decrease or eliminate the force exerted on the tibia. Specifically, the user presses the center of the top cap 199 over the center aperture 197 of the knob 196 to exert a force against the retaining ring 189. The force on the retaining ring 189 moves the drive shaft 130 along the axis A-A in a direction Y and disengages the teeth 132 (FIG. 3) of the drive shaft 130 from the first teeth 171 (FIG. 3) of the center gear 170. When the teeth 132 and 171 are disconnected, the gears 170 and 175 and spool 160 can rotate freely and independently of the drive shaft 130. Accordingly, while pressing the top cap 199, the user can feed cable 111 out of the tensioning mechanism 120 to reduce the tension in the assembly 110.

One feature of the illustrated tensioning mechanism 120 is that the clutch 180 inhibits the clutch hex 190, drive shaft 130, and spool 160 from rotating about the axis A-A in the direction E, unless a user exerts a force against the center of the top cap 199 as described above. An advantage of this feature is that the tension in the cable 111 and, consequently, the force exerted on the tibia remains consistent over time and is independent of the position of the upper and lower frames 102 and 104.

Another feature of the illustrated knee brace 100 is that the knob 196 allows a user to dial-in a precise, desired force while the brace 100 is worn on the leg. Accordingly, unlike many prior art knee braces, the illustrated brace does not need to be removed from the user's leg to adjust the force. An advantage of this feature is that the user saves the time and hassle of removing the brace, adjusting the force, donning the brace back onto the leg, determining if the adjusted force is the desired force, and if not, repeating the process. Another advantage of the illustrated knee brace 100 is that the user can more easily fine-tune the precise, desired force applied to the tibia because the user can feel the force while adjusting the tension of the cable 111.

Another feature of the illustrated knee brace 100 is that as the tension in the cable 111 increases, the segments of the cable 111 in the cable guide 113 move toward the user and exert a force on the user's leg. As such, the segments of the cable 111 between the first and second pulleys 112a-b and in the cable guide 113 exert a force on the tibia. This force is exerted over a “T” shaped area of the tibia. An advantage of this feature is that assembly 110 exerts a force over a larger region of the tibia, which provides better contact and support for the tibia.

From the foregoing, it will be appreciated that specific embodiments of the invention have been described herein for purposes of illustration, but that various modifications may be made without deviating from the spirit and scope of the invention. For example, in several embodiments, the brace can include a tensioning mechanism having a different configuration than that described above for manually adjusting the tension of the cable in the assembly. Accordingly, the invention is not limited except as by the appended claims.

1. A brace for use on a limb of a user, the brace comprising:
a first frame portion;
a second frame portion;
a hinge movably coupling the first frame portion to the second frame portion;
a flexible member positioned relative to the first and/or second frame portion for exerting a force on the limb of the user, the force being at least generally independent of the position of the first frame portion relative to the second frame portion; and
a tensioning mechanism for manually adjusting a tension in the flexible member to vary the force exerted on the limb of the user.

2. The brace of claim 1 wherein:
the flexible member comprises a cable and/or filament; and
the tensioning mechanism comprises a roller for winding the cable and/or filament, a driving member for rotating the roller in a first direction, and a clutch for selectively inhibiting rotation of the roller in a second direction opposite the first direction.

3. The brace of claim 1 wherein the flexible member comprises a cable and/or filament.

4. The brace of claim 1 wherein the flexible member is positioned to exert the force over a “T” shaped area of the limb of the user.

5. The brace of claim 1 wherein:
the flexible member comprises a cable and/or filament; and
the brace further comprises first and second pulleys attached to the second frame portion with the cable and/or filament extending between the first and second pulleys.

6. The brace of claim 1 wherein the tensioning mechanism comprises a driving member coupled to the flexible member for adjusting the tension in the flexible member.

7. The brace of claim 1 wherein the tensioning mechanism is configured to adjust the force exerted on the limb of the user without the user manipulating a strap on the brace.

8. The brace of claim 1 wherein the tensioning mechanism is configured to adjust the force exerted on the limb of the user without the user removing the brace from the limb.

9. The brace of claim 1 wherein the brace is a knee brace and the flexible member is positioned on the knee brace to exert the force on the tibia of the user.

10. The brace of claim 1 wherein:
the flexible member comprises a cable and/or filament; and
the brace further comprises a roller for winding the cable and/or filament to adjust the tension in the cable and/or filament.

11. A knee brace for exerting a force on a tibia of a user, the knee brace comprising:
an upper frame;
a lower frame;
a hinge movably coupling the lower frame to the upper frame;
first and second guides attached to the lower frame;
a cable and/or filament having a segment extending between the first and second guides and positioned to selectively exert a force on the tibia of the user; and
a tensioning mechanism attached to the lower frame, the tensioning mechanism including a driving member for manually adjusting the tension in the cable and/or filament to change the force exerted on the tibia of the user.

12. The knee brace of claim 11 wherein the tensioning mechanism and the cable and/or filament are configured so that the force exerted on the tibia is at least generally independent of the position of the upper frame relative to the lower frame.

13. The knee brace of claim 11 wherein the first and second guides comprise first and second pulleys.

14. The knee brace of claim 11 wherein the cable and/or filament is positioned to exert the force over a “T” shaped area on the tibia of the user.

15. The knee brace of claim 11 wherein the tensioning mechanism further comprises a roller for winding the cable and/or filament and a clutch operably coupled to the roller, wherein the driving member is configured to rotate the roller in a first direction to increase the tension in the cable and/or filament, and wherein the clutch selectively inhibits rotation of the roller in a second direction opposite the first direction.

16. A brace for use on a limb of a user, the brace comprising:
a first frame portion;
a second frame portion;
a hinge movably coupling the first frame portion to the second frame portion;
a cable and/or filament positioned relative to the first and/or second frame portion for exerting a force on the limb of the user; and
means for manually adjusting a tension in the cable and/or filament.

17. The brace of claim 16 wherein the cable and/or filament is configured to exert a generally consistent force on the limb of the user independent of the position of the first frame portion relative to the second frame portion.

18. The brace of claim 16 wherein the means for manually adjusting the tension in the cable and/or filament comprise a roller for winding the cable and/or filament, a driving member for rotating the roller in a first direction, and a clutch for selectively inhibiting rotation of the roller in a second direction opposite the first direction.

19. The brace of claim 16 wherein the means for manually adjusting the tension in the cable and/or filament comprise a driving member coupled to the cable and/or filament for adjusting the tension in the cable and/or filament and the force exerted on the limb of the user.

20. The brace of claim 16 wherein the cable and/or filament is positioned to exert the force over a “T” shaped area on the limb of the user.
